SEQUENCE LISTING (14 sequences)

SEQ ID NO: 1 - protein, 16 aa, Homo sapiens
ADSGEGDFLAEGGGVR

SEQ ID NO: 2 - protein, 10 aa, Homo sapiens
RIHWESASLL

SEQ ID NO: 3 - protein, 11 aa, Homo sapiens
HRIHWESASLL

SEQ ID NO: 4 - protein, 12 aa, Homo sapiens
THRIHWESASLL

SEQ ID NO: 5 - protein, 13 aa, Homo sapiens
ITHRIHWESASLL

SEQ ID NO: 6 - protein, 14 aa, Homo sapiens
KITHRIHWESASLL

SEQ ID NO: 7 - protein, 15 aa, Homo sapiens
SKITHRIHWESASLL

SEQ ID NO: 8 - protein, 16 aa, Homo sapiens
SSKITHRIHWESASLL

SEQ ID NO: 9 - protein, 17 aa, Homo sapiens
SSKITHRIHWESASLLR

SEQ ID NO: 10 - protein, 16 aa, Homo sapiens
SKITHRIHWESASLLR

SEQ ID NO: 11 - protein, 11 aa, Homo sapiens
THRIHWESALL

SEQ ID NO: 12 - protein, 7 aa, Homo sapiens
IHWESLL

SEQ ID NO: 13 - protein, 21 aa, Homo sapiens
TMSILDISMMTGFAPDTDDLK

SEQ ID NO: 14 - protein, 9 aa, Homo sapiens
SHVSELLML

(19) United States
(12) Patent Application Publication
(10) Pub. No.: US 2004/0121306 A1
(43) Pub. Date: Jun. 24, 2004

(54) Method of confirming the presence of myocardial infarction

(76) Inventors: Peter Kupchak, Toronto (CA); George Jackowski, Kettleby (CA); John Marshall, Toronto (CA)

Correspondence Address: McHale & Slavin, P.A., Suite 402, 44-40 PGA Blvd., Palm Beach Gardens, FL, 33410, US

(21) Appl. No.: 10/325,162

(22) Filed: Dec. 20, 2002

(51) Int. Cl. C12Q 1/00

(52) U.S. Cl. 435/4; 702/19

(57) ABSTRACT

The mass distribution and intensity of polypeptides from human sera obtained using mass spectrometry has served as a fingerprint used to diagnose disease. The instant invention relates to a method for diagnosing and distinguishing a myocardial infarction in a particular human subject by comparing a serum protein profile of the particular human subject to the reference serum protein profiles of at least two or more defined subsets of human subjects wherein said serum protein profiles are generated by mass spectrometry comprising the steps of; identifying areas of the serum protein profiles that are different in signal intensity; reducing the dimensionality of the areas identified; elucidating a metric, and analyzing the data elucidated in order to diagnose and distinguish a myocardial infarction in a particular human subject.

### FIELD OF THE INVENTION

The instant invention relates to the field of proteomics, particularly to the analysis of proteins using mass spectrometry in order to diagnose disease and other physiological conditions, and most particularly to the use of statistical methods to compare serum protein distributions in samples analyzed by mass spectrometry and to diagnose disease and other physiological conditions based upon differences in the serum protein distributions between the samples.

### BACKGROUND OF THE INVENTION

There has been an explosion in interest in the use of mass spectrometry (MS) to diagnose disease and other physiological conditions (Howard et al. Applied Environmental Microbiology 66(10):4396-4000 2000; Paweletz et al. Disease Markers 17(4):301-307 2001; Ardekani et al. Expert Rev Mol Diagnostics 2(4):312-320 2002). To date the field has focused on the use of highly sensitive but low-resolution MALDI-TOF1 mass spectrometers termed SELDI-TOFs to record spectra of the low molecular mass proteins and polypeptides in sera or biological fluids. The spectral patterns act as fingerprints that are mathematically analyzed to identify the sample as belonging to a certain physiological condition. Consensus is emerging that these peptides profiles have great utility as diagnostics. However, since the peptides and small proteins that form these patterns have not been identified there has been to date been no understanding of what mechanisms produce the spectral patterns. Mass spectrometric profiling of the low molecular mass peptides in blood or other biological fluids may provide a means to diagnose many diseases and physiological conditions in man. The mass spectral diagnostic technique is simple and inexpensive to develop into a working assay and similar laboratory methods can be used to discriminate between a variety of diseases without specialized reagents for each condition under study. Hence mass spectrometry may provide methods to detect and discriminate between so-called orphan or rare diseases where traditional diagnostics have not been economical to develop.

In contrast to genetic testing for known sequences that may indicate the propensity to develop disease, mass spectral diagnosis may be able to detect the manifestation of the phenotype itself without knowledge of the genetic lesion(s) involved. Computer assisted classification of the mass spectra requires no characterization of genetic materials. This may in turn obviate many ethical concerns regarding genetic diagnostic technologies. Computer assisted analysis of mass spectra as an aid to diagnosis has been reported using a two step learning algorithm for the comparison of SELDI-TOF MS spectra of biological fluids (Ball et al. Bioinformatics 18(3):395-404 2002). Alternatively, quantitative decision tree analysis of mass spectra has been employed (Qu et al. Clincial Chemistry 48(10):1835-1843 2002). Indeed, in 2002 an international conference and competition was held at Duke University dedicated to the mathematical discrimination of subtle differences between MALDI-TOF spectra. Parenthetically, the job of the biochemist in this enterprise is to find sample preparations that reveal large differences between treatments and thus avoid elaborate mathematical treatments. The instant inventors report the use of a combination of quantitative decision analysis combined with multivariate analysis to provide a statistically appropriate and powerful method of comparing peptide distributions. When carrying out the methods of the instant ivention, it is noted that conceptually there is no need to limit the mass spectrometer employed to MALDI or SELDI mass spectrometers. Theoretically, any MS experiment including LC-MS or CE MS experiments may be used to discriminate between disease states. Currently, there is intense interest in mass spectral diagnosis based on spectral profiling of the families of low molecular mass proteins polypetides in sera or plasma. However to date no one has shown the mechanism underlying the presence of the diagnostic peptides in blood.

While many papers have now shown the existence of different polypeptides in biological fluids from various disease states, no one has identified the peptides. Within the last decade the Edmund degradation method that was typically used for the identification of unknown proteins has been complemented by rapid advances in the use of ESI and MALDI followed MS/MS fragmentation (de Hoffmann and Stroobant, 2001, pp239-275). In the method of the instant invention two kinds of MS/MS analysis; LC-ESI-ION TRAP and MALDI-Qq-TOF (Griffin et al. Analytical Chemistry 73(5):978-986 2001) were used to identify some of the peptides in the fingerprint. MS/MS analysis is tandem mass spectrometry where the first mass analyzer isolates a certain peptide, the peptide is fragmented by acceleration through an electric field in the presence of homonuclear gas molecules producing CID fragmentation, and the fragments are analyzed by a second mass analyzer that records the peptide fragmentation spectra. That spectra is then compared using a computer to the predicted fragmentation pattern of proteins encoded by the human genome, cDNA banks and EST data. In the case of MALDI-Qq-TOF (Loboda et al. Rapid Commun Mass Spectrometry 14(12):1047-1057 2000; Shevchenko et al. Analytical Chemistry 72(9):2132-2141 2000), the a quadrupole mass analyzers Q was connected in series to a TOF analyzer via a radio-frequency only quadropole q that acts as a trap to fragment the peptide of interest. In the case of the ION-TRAP (Link et al. Nature Biotechnology 17(7):676-682 1999) the same mass analyzer is used to isolate and collect the analyte, fragment the peptide, and then record the fragmentation spectra. Thus, these three function are separated in time instead of space as is the case with other tandem mass spectrometers. The computational identification of proteins based on mass spectrometry is sometimes termed proteomics (Mann et al. Annual Review of Biochemistry 70:437-473 2001).

Heart attack, also known as myocardial infarction (MI), is a major cause of death in men and women especially in western society (Azzazy et al. Clinical Biochemistry 35(1):13-27 2002; Rogers et al. Current Cardiol Rep 4(4):341-347 2002). Yet cardiac distress remains difficult to diagnose definitively by standard methods such as troponin I test without specialty reagents and optimized assays (Ellestad et al. Cardiology 93(4):242-248 2000; Douketis et al. Archives of Internal Medicine 162(1):79-81 2002). It remains possible that the victims of massive heart attacks have been previously suffering minor events that have gone undiagnosed. Since heart attacks can largely be avoided or delayed by altering diet and exercise, and since effective treatment may be required, instant diagnosis is particularly germane to develop reliable diagnostics for MI. Hence, the instant inventors applied mass spectral diagnostic techniques to rapidly develop diagnostic assays for MI.

A side effect of the deeper mass spectral exploration of diagnostic peptide patterns may well be new insights into the cause of disease or disease symptoms. For example, mounting evidence indicates that the damage done to the heart after MI results from an inflammatory reaction resulting in damage to the myocardial tissue that is mediated by complement driven cellular attack (Frangogiannis et al. Cardiovascular Research 53(1):31-47 2002). The beginning of the inflammatory response may include the recruitment of the early complement factor C3 to the site of necrotic cells. C3 precursor is cleaved to form the C3 beta chain and the C3 alpha chain. The C3 beta chain remains intact. The C3 alpha chain contains the anaphylatoxin, the thioester site that may attach to cellular surfaces via the thioester link and direct the activation of white blood cells (Sahu et al. Immunology Review 180:35-48 2001; Nielsen et al. Journal of Leukocyte Biology 72(2):249-261 2002), and a variety of cleavage sites that result in the progressive processing of the molecule to form C3a, C3b, iC3b or result in the generation of fragments including C3c, C3DG, C3D, or C3f.

Before the rise in interest in the use of mass spectrometry to diagnose disease and other physiological conditions, prior artisans have experimented with techniques such as, cell fingerprinting (Zhou et al. Proteomics 1(5):683-690 2001). Zhou et al. developed a statistical framework for classifying cells according to the set of peptide masses obtained by mass spectrometric analysis of digests of whole cell protein extracts. Zhou et al. used defined bacterial strains to test their approach. A mass spectrometric analysis was repeated for extracts obtained at different points in the growth curve in order to define an invariant set of signals(representative of protein masses) that uniquely identify the bacterium. In contrast to the instant invention the method of Zhou et al. was performed using digests of the protein content of an entire cell to identify that particular cell not serum proteins which represent products of many cellular types to identify a particular disease state. Additionally, the method, of Zhou et al. utilizes protein digestion with known enzymes showing a known pattern as opposed to the instant invention which seeks to identify enzymatic changes in serum proteins as representative of a disease state.

The instant inventors found in agreement with prior art (Paweletz et al. Disease Markers 17(4):301-307 2001; Ardekani et al. Expert Rev Mol Diagnostics 2(4):312-320 2002) that a high-sensitivity, low mass accuracy form of MALDI-TOF (Merchant et al. Electrophoresis 21(6):1164-1177 2000; Weinberger et al. Pharmacogenomics 1(4):395-416 2000)could be used to rapidly generate serum peptide fingerprints that distinguish between disease states. However, what is lacking in the art is a method to rapidly interpret and assign a disease state to serum protein profiles generated by mass spectrometry that is simple, economical and does not require specialized reagents or optimized assays. The statistical analysis of the mass spectral patterns by a method that is both quantitative, rigorous and statistically powerful will be a key component of this type of anaylsis (Fung et al. Biotechinques Supplement 34-38 and 40-41 2002; Li et al. Clinical Chemistry 48(8):1296-1304 2002). Multivariate analysis is a powerful method for contrasting populations that lacks a quantitative element and thus might differentiate between groups of peaks that only show modest real differences. Hence the instant inventors inserted a quantitative cut-off in signal intensity similar in concept to decision tree analysis (Qu et al. Clinical Chemistry 48(10):1835-1843 2002), and thus only analyzed data where the media intensities in each 5D window differed by at least a factor of two. By adjusting the stringency factor prior to multivariate analysis even greater levels of confidence might be obtained. In the data sets shown herein, where over 50 different sets of peak intensities were used to contrast samples very convincing probabilities were associated with each. Thus by combining a quantitative decision step to ensure that the set of scalar values subjected to analysis are markedly different between treatments prior to inclusion in the metric of data used for subsequent multivariate analysis will ensure that these approaches are robust, reliable and powerful. The instant inventors have provided a method for diagnosing and distinguishing a myocardial infarction in a particular human subject by comparing the serum protein profiles of the particular human subject to the reference serum protein profiles generated by mass spectrometry that is simple, economical does not require specialized reagents or optimized assays.

### SUMMARY OF THE INVENTION

The mass distribution and intensity of polypeptides from human sera obtained using mass spectrometry has served as a fingerprint used to diagnose disease. However, the mechanism underlying mass spectral diagnosis has not been demonstrated. The instant invention relates to a method for diagnosing and distinguishing a myocardial infarction in a particular human subject by comparing a serum protein profile of the particular human subject to the reference serum protein profiles of at least two or more defined subsets of human subjects wherein said serum protein profiles are generated by mass spectrometry comprising the steps of; identifying areas of the serum protein profiles that are different in signal intensity; reducing the dimensionality of the areas identified; elucidating a metric, and analyzing the data elucidated in order to diagnose and distinguish a myocardial infarction in a particular human subject. The instant inventors have described a method useful to obtain a serum fingerprint of myocardial infarction patients which forms the basis of a powerful diagnostic tool that rapidly and conclusively confirms the presence of myocardial infarction. This method shows that in general mass spectral diagnosis works on the principal of detecting post-translational modifications of major serum proteins effected by disease associated activities in the blood. In the case of myocardial infarction, the MALDI-TOF spectra of peptides collected by C18 reversed-phase chromatography form a diagnostic pattern resulting from the post-translational modification of complement C3 alpha chain to release the C3f fragment and cleavage of fibrinogen to release the alpha peptide. Time course and PMSF studies were used to demonstrate that the peptides that form diagnostic patterns in the serum result from polypeptides that were continually being generated by serine-centered endo-proteinases. However, it is also shown that the peptides cleaved from serum proteins by endo-proteinases are themselves in turn degraded by N-terminal exopeptidase(s), i.e. aminopeptidase. Thus the mass spectral patterns that form the basis of diagnosis reflects a balance of the proteinase and aminopeptidase specificity and activity in the sera. On this basis MALDI-TOF, or other mass spectra diagnostics, of sera may reflect the interaction of disease-associated molecules with the proteins of the blood.

Accordingly, it is an object of the instant invention to provide a method for diagnosing and distinguishing a myocardial infarction in a particular human subject by comparing a serum protein profile of the particular human subject to the reference serum protein profiles of at least two or more defined subsets of human subjects wherein said serum protein profiles are generated by mass spectrometry comprising the steps of; identifying areas of the serum protein profiles that are different in signal intensity; reducing the dimensionality of the areas identified; elucidating a metric, and analyzing the data elucidated in order to diagnose and distinguish a myocardial infarction in a particular human subject.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

FIG. 1. shows a high-level classification methodology flowchart which outlines the steps of the instant invention.

FIG. 2. shows serum protein profiles from six selected reference subjects.

FIG. 3. shows serum protein profiles from six selected heart attack subjects.

FIG. 4. shows plots of median intensities at each selected protein mass for reference and heart attack subjects.

FIG. 5. is Table 1 showing the classification results from the first set of experiments.

FIGS. 6A-B. 6A is Table 2 showing the posterior probabilities of classification of reference and MI subjects from the second set of experiments (significance level of α=0.001). 6B is Table 3 showing the posterior probabilities of classification of reference and MI subjects from the second set of experiments (significance level of α=0.005).

FIG. 7. shows the N-terminal deletion series of the alpha fibrinogen peptide obtained from the sera of MI patients using LC-ESI-ION-TRAP. The sequences shown are from top to bottom; SEQ ID NO:1, residues 2-16 of SEQ ID NO:1, residues 3-16 of SEQ ID NO:l, residues 4-16 of SEQ ID NO:1, residues 5-16 of SEQ ID NO:1, residues 6-16 of SEQ ID NO:1, residues 7-16 of SEQ ID NO:1 and residues 7-15 of SEQ ID NO:1.

FIG. 8. shows the comparison of mass spectra of reference and MI serum samples resolved on the Cyphergen Biosystems PBS II MALDI-TOF.

FIGS. 9A-B. shows the distribution of peptides between normal and MI serum that differ in median intensity by an average of two fold. 9A shows a significance level of α=0.001 and 9B shows a significance level of α=0.005.

FIGS. 10A-B. shows the identity of peptides in the reference and MI sera as determined by a MALDI-Qq-TOF using an interface to a Micro Mass mass spectrometer related to MALDI-TOF spectra obtained with the Cyphergen Biosystems PBS II. 10A shows reference serum, H indicates that the peptide is derived from human serum albumin. 10B shows MI serum, C and A indicate that the peptide is derived from complement C3f and fibrinogen alpha peptide respectively.

FIG. 11. shows an example of an N-terminal deletion series of the C3f fragment of complement C3 as determined by MALDI-Qq-TOF. The sequences shown are from left to right, SEQ ID NOS:2-9.

FIG. 12 shows a western blot against the complement C3 alpha chain from reference and MI sera pre-fractionated with DEAE sepharaose.

FIG. 13. shows a CBBR stained gel of sera from reference and MI sera pre-fractionated with DEAE sepharaose.

FIG. 14. shows a summary of mass spectral data collected concerning complement C3 post translational processing.

FIG. 15. shows the effect of time on the MALDI-TOF spectra of normal human plasma.

FIG. 16. shows the effect of the iron-sulphur protease inhibitor EDTA and the serine-centered protease inhibitor PMSF on the MALDI-TOF spectra of normal human sera.

FIG. 17. shows the interaction of time and PMSF on the MALDI-TOF spectra of normal human sera.

FIG. 18. shows the effect of serine endo proteinase inhibition on the MALDI-TOF spectral pattern of normal and MI sera.

### LIST OF ABBREVIATIONS AND DEFINITIONS

The following abbreviations are used throughout the instant specification:

C3 is complement C3; C3f is the fragment f of complement C3; CID is collision induced decay; CBBR is coomasie brilliant blue; CHCA is cyano-4-hydroxy cinnamic acid; LC-ESI-MS is liquid chromatography-electroionspray ionization mass spectrometry; MALDI-TOF matrix assisted laser desorption and ionization time of flight spectrometry; MI is myocardial infarction or myocardial infarct; MS is mass spectrometry; MS/MS is tandem mass spectrometry; NHS is normal human sera; SELDI-TOF is surface enhanced laser desorption and ionization time of flight spectrometry.

The terms myocardial infarct, myocardial infarction and heart attack are used herein interchangeably.

The terms “reference sera” and “control sera” are used herein interchangeably.

As used herein, the term “defined subsets” refers to a group of the serum protein profiles of reference subjects having confirmed clinical diagnoses.

As used herein, the term “dimensionality” refers to the number of data points seen on a mass spectral analysis.

As used herein, the phrase “areas of a spectrum” or “area of a serum protein profile” refers to portions of a mass spectrum having one or more data points.

### DETAILED DESCRIPTION OF THE INVENTION

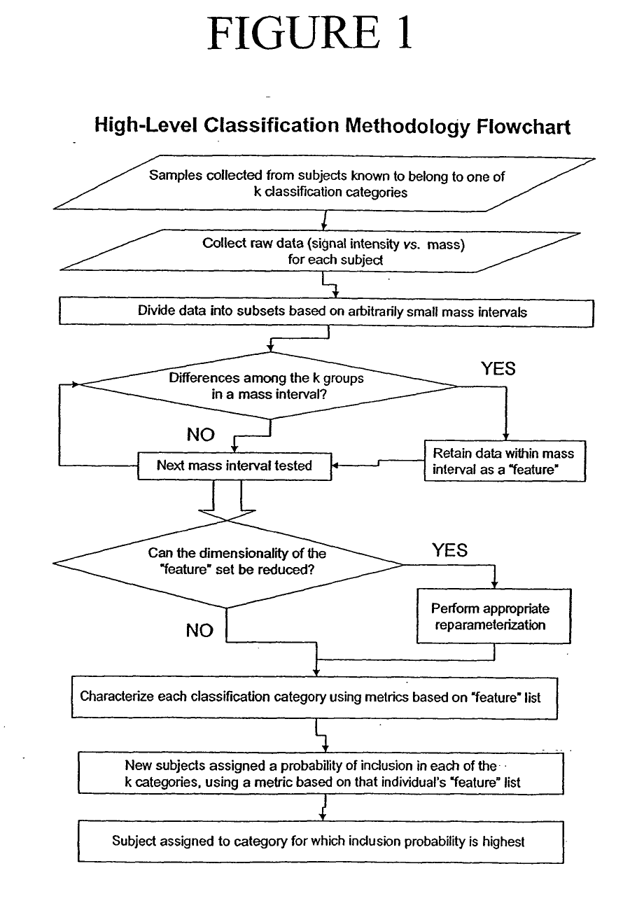

The instant invention presents a novel methodology for classifying human subjects on the basis of their serum protein profiles (see FIG. 1 for the methodology flowchart). The methodology uses serum protein profiles available for two or more well-defined subsets of human subjects with confirmed clinical diagnoses or other such confirmed classification labels, for example, but not limited to, “normal” vs. “diseased”; “low-risk” vs. “moderate-risk” vs. “high-risk”. Particular human subjects to be classified according with the instant methodology should have their serum protein profile performed under identical conditions to those conditions used for the well-defined subsets of subjects.

The serum protein profiles are of a very high dimensionality, meaning that a spectrum from a human serum sample can consist of thousands of data points. Thus, it canbe difficult to ascertain information regarding one particular protein represented by one particular data point among thousands. The steps of the instant methodology can be grouped into three stages. Stage One of the methodology involves identification of areas of the mass spectra which are demonstrably different between the reference subjects and the test subjects (particular human subjects) in terms of signal intensity (each intensity represents a specific protein mass) A statistical decision rule is utilized in order to determine whether the signals associated with a specific protein mass are significantly different among the subsets of subjects. Stage Two of the methodology involves reducing the dimensionality of the data so that only the signal intensities associated with protein masses identified in Stage One for each subject in the subsets is retained. Stage Three of the methodology involves the construction of a metric used to identify new subjects in the test subset, based on the statistical characteristics of the data associated with subjects who are known to belong to each of the well-defined subsets. The data generated by the metric is analyzed and compared to diagnose or distinguish the desired classification, or disease state, such as a myocardial infarction as is exemplified in the instant examples. Probabilities are computed at the end of Stage Three to reflect the likelihood that a new test subject is classified in each of the given subsets, in order to provide an indication of the confidence which one has in assigning a given classification label to the subject.

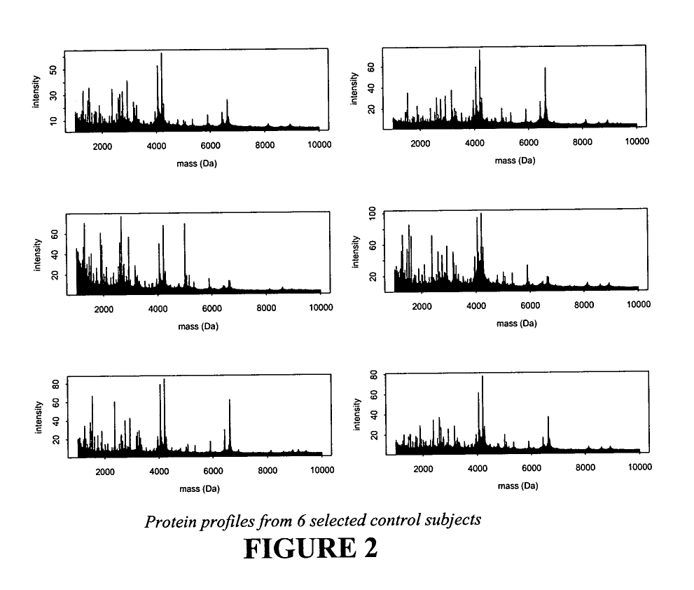

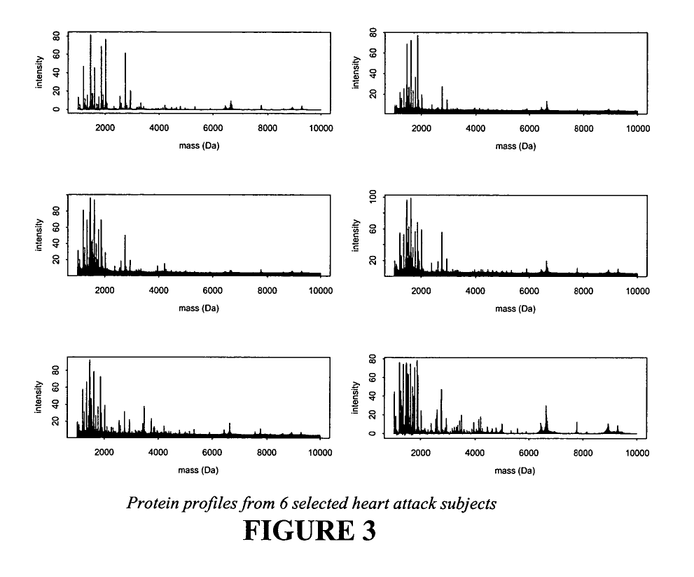

The first experiment shows the stages of the methodology. FIGS. 2 and 3 show serum protein spectral profiles of reference subjects and test subjects respectively. In stage one (identification of areas), the data was split into mass intervals with each interval having a width of approximately five Daltons. The mean intensity of heart attack subjects was compared to that of control subjects within each mass interval, using a two-way analysis of variance (ANOVA) and treating the individual masses as blocks. Within each mass interval, the F-statistic for testing whether the mean intensity of the heart attack subjects equals that for the reference subjects was compared.

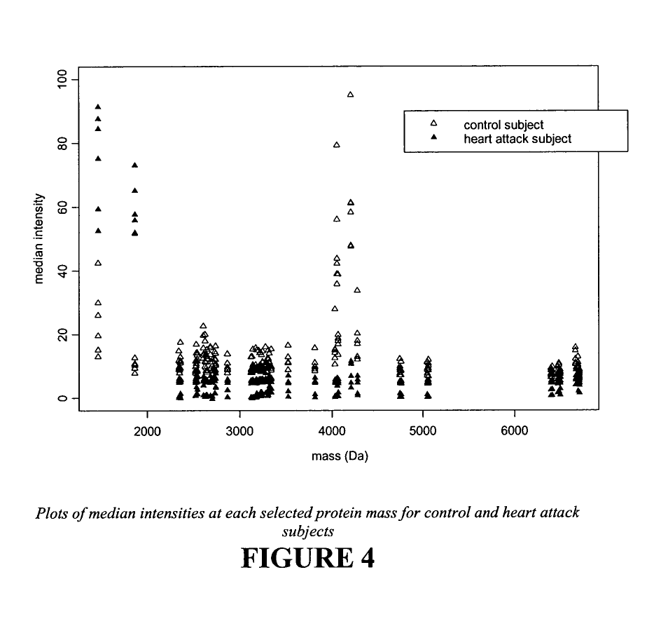

In stage 2 (reducing the dimensionality), the data for all mass intervals with an associated F-statistic of greater than 200 was retained. The mid-point of each of these mass intervals and median intensity for each subject within each of these mass intervals was computed. FIG. 4 shows a plot of median intensities for each reference and each heart attack subject within each selected mass interval, a total of 51 such intervals were selected at this stage.

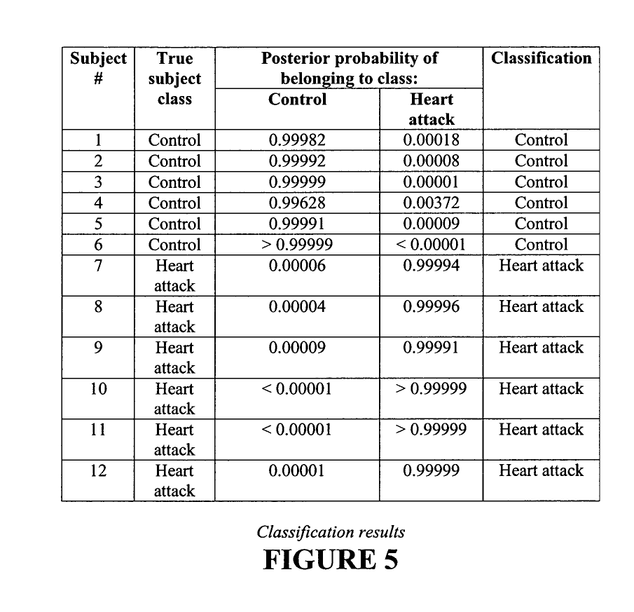

In stage three(constructing a metric), a linear discriminant analysis was employed using the technique of “leave one out” cross-validation in order to classify each subject as either a reference subject or a heart attack subject, based on the relative proximity of the vector of intensities for that subject to the mean vectors of intensities for the subject in question. In addition, the posterior probability of assigning the subject to each of the two classes was computed using an application of Bayes' Rule. The results are shown in the Table of FIG. 5.

A second set of experiments was carried out to further test the methodology. Except where indicated all dry chemical were obtained from the Sigma chemical company and were of a fine grade (St Louis, Mo.). All solvents were of an optical grade or better. DEAE chromatographic resin was obtained from BIORAD laboratories. Reversed phase resin was obtained from Millipore laboratories. The C3b alpha chain antibody was obtained from x.

Blood Samples

Blood samples were obtained under a human ethics protocol. The blood from MI patients was drawn within 2H (2 hours) of the suspected event and in each case the presence of heart attack was confirmed by standard methods (Ellestad et al. Cardiology 93(4):242-248 2000; Douketis et al. Archives of Internal Medicine 162(1):79-81 2002). The protocol ensures that plasma or sera is collected and stored at room temperature for no more that 2h before freezing. Blood samples were drawn into citrated tubes for plasma and into standard tubes for sera. The samples were thawed, aliquoted and re-frozen once before being used and discarded. In this experiment the principle of post translational modification of sera proteins was demonstrated using the sera of six randomly selected MI and six randomly selected normal patients.

MALDI MS

Sera for MALDI-TOF analysis was diluted ten fold in 0.1% TFA. Typically 10 micro litres are used for the analysis. The peptides were collected in a batch modes by passage over C18 reversed phase resin washed with several column volumes of 0.1% TFA and eluted with 50% acetonitrile in water with 0.1% TFA and 5% formic acid. The eluted peptides were dried on to gold MALDI targets. After allowing all the samples to dry evenly an energy absorbing molecule or matrix was applied. A few milligrams of the matrix CHCA was deposited into a mass spec compatible sample tube, the matrix was washed by re-suspension in 50% acetonitrile in 0.1% TFA in water before discarding the wash solution and covering the matrix with fresh 50% acetonitrile 0.1% TFA to from a saturate solution. One microlitre of saturated matrix solution was applied to each MALDI target spot immediately before sampling. The data was collected using a TOF MS model PBSII provided by Cyphergen (Weinberger et al. Pharmacogenomics 1(4):395-416 2000).

Statistical Analysis of MALDI-MS Spectra Analytes detected in MALDI-MS spectra were categorized into 5 D windows. Within each window, a nested factor design arrangement was assumed, with the type of group (MI vs. control) considered a fixed effect, and six random individual subjects nested within each group. The appropriate F statistic associated with the null hypothesis of equality in mean signal strength between the MI group and the control group was computed within each 5 D window, by dividing the mean square associated with the type of group by the mean square associated with subjects nested within a group. Significance levels of a=0.005 and a=0.001 were considered for the analysis; with respect to the windows which showed extreme differences between the two groups (i.e. where the p-value associated with the F test of interest was smaller than the appropriate significance level), the median signal strength for each subject was computed and retained for subsequent multivariate analysis. Specifically, a linear discriminant analysis was employed using the technique of “leave one out” cross-validation in order to classify each subject as either a control subject or an MI subject, based on the relative proximity of the vector of signal strengths for that subject to the mean vectors of signal strengths for the two classes of subjects (previously computed by leaving out the vector of signal strengths for the subject in question). In addition, the posterior probability of assigning the subject to each of the two classes was computed using an application of Bayesian methodology (Krzanowski and Marriot,1995). All statistical analyses were performed using S-PLUS Version 6 software for Windows (Insightful Corporation, Seattle, Wash.).

MS/MS by MALDI-Qq-TOF

Polypeptides from sera samples were prepared for MALDI analysis as described above but spotted on a gold chip suitable for a Micromass optima MALDI-Qq-TOF. MS/MS spectra were collected using CHCA as a matrix. The fragment patterns were searched against a non-redundant library of DNA, cDNA's EST and proteins assembled from publically available data in September 2002. The MS/MS fragmentation patterns were searched against the data bases using MASCOT. Only MS/MS spectra with significant Mouse scores are reported as previously described (Chu et al. Analytical Chemistry 74(9):2072-2082 2002; Kottis et al. Journal of Neurochemistry 82(6):1566-1569 2002).

MS/MS by LC-ESI-ION TRAP

Peptides were collected from sera using batch reversed phase chromatography by dilution 10 fold in 0.1% TFA in water before collection over C18 resin, washing in at least three column volumes and elution with a final v/v/ of 0.1% TFA and 50% acetonitrile before drying, re-dissolving in 0.1% TFA in water and separation C18 revered phase chromatography (Agilent 0.3 mm ID, 15 cm column). The sample was analyzed over a 90 minute gradient from 5% to 65% acetonitrile at a flow rate of 1 microlitre per minute with an Agilent 1100 series capillary pump through a VYDAK 150×0.3 mm C18 column via a metal needle electro-spray head at 3000 volts into a Decca XP ION TRAP (Finnegan). The resulting MS/MS spectra were analyzed by SEAQUEST. Peptides identified by SEAQUEST with significant X-correlations were reported as previously described (Washburn, et al. Nature Biotechnology 19(3):242-247 2001).

DEAE Chromatography, SDS PAGE and Western Blot

DEAE columns were fritted with glass wool and eqillibriated with binding buffer (20 mM pH 8.5 tricine 100 mM NaCl). 25 ul samples from control and MI patients were mixed with 500 ul of 20 mM pH 8.5 tricine 100 mM NaCl and passed over the column. The column was washed in 500 ul of binding buffer before eluting in 200 ul of 20 mM pH 8.5 tricine 500 mM NaCl. For SDS-PAGE analysis the protein eluted from DEAE were mixed with an equal volume of 2× sample buffer and were resolved on 7% tricine gels (Schagger et al. Analytical Biochemistry 166(2):368-379 1987). The gels were either stained with CBBR for protein sequencing or transferred to PVDF for western blots. For western blots gels were electrotransfered onto PVDF methanol-glycine buffer (Towbin et al. PNAS USA 76(9):4350-4354 1979), blocked in 5% skim milk powder before incubating with mouse anti C3b alpha and detected with Goat anti mouse HRP (Jackson Laboratory) using ECL (Amersham-Pharmacia) on Kodak ECL film. For protein sequencing, the gel was stained with CBBR in 40% methanol and 10% acetic acid before the indicated band cut from the gel and trypsinized (Kottis et al. Journal of Neurochemistry 82(6):1566-1569 2002) by MALDI-Qq-TOF (PE SCIEX, QSTAR pulsar i) and LC-ESI-ION TRAP (Finnegan, Decca XP).

Proteolysis of Sera and Plasma Samples

Fresh plasma samples for time course studies were collected in citrated tubes, and immediately centrifuged at 14,000 g for 30 seconds, the supernatant was collected the plasma diluted 1 to 10 in 0.1 % TFA. For time course studies the plasma was left the bench for the times indicated in the results, before quenching with 10 volumes of 0.1% TFA. PMSF was dissolved in a 0.5 M stock solution in DMSO immediately before use. PMSF was added at concentrations as high as 5 mM with the appropriate amount of the DMSO alone added to the controls. Alternatively the PMSF was weighed out and added directly to the serum with similar results. The reaction was permitted to proceed for the times indicated in the results before quenching in 0.1% TFA.

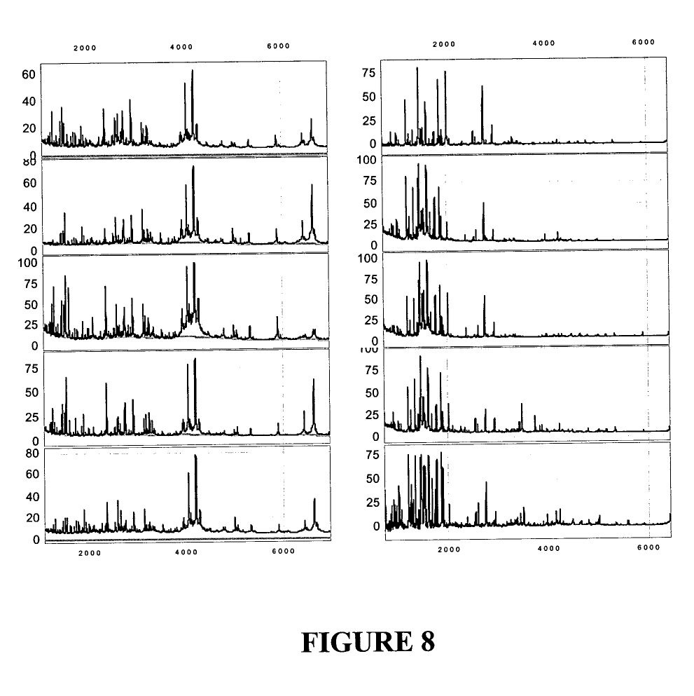

By enriching peptides from acidified sera with C18 resin and analyzing the results with a MALDI-TOF it seems possible to discriminate between physiological states. The Cyphergen Biosystems MALDI-TOF is a single flight path TOF that has been designed to sacrifice resolution and mass accuracy in favor of sensitivity. For example, FIG. 8 shows distinctive variations in patterns between normal human sera (NHS) and Myocardial Infarction (MI), i.e. Heart Attack. FIG. 8 shows the comparison of mass spectra of control and myocardial infarction serum samples resolved on the Cyphergen Biosystems PBS II MALDI-TOF. The peptides from 10 ul of sera were collected by batch reversed phase chromatography and eluted onto the targets spots of gold chips and matrixed with 1 microlitre of saturated CHCA matrix immediately before analyzing at a laser intensity of 200 and a sensitivity setting of seven. Five representative spectra of control (left of FIG. 8) and MI (right of FIG. 8) patients are shown. The result seems in agreement with previous artisans who also found distinctive patterns in the sera of cancer patients (Paweletz et al. Disease Markers 17(4):301-307 2001; Ardekani et al. Expert Rev Mol Diagnostics 2(4):312-320 2002). However, compared to the previously published results, pre-fractionating sera by collection of the peptides by reversed phase chromatography prior to MALDI-TOF analysis produced exceptionally clear differences between normal human sera and MI sera. FIG. 8 shows the reproducibility the method with 5 different NHS and MI samples ionized with CHCA matrix. Similar trends were obtained when sinipinic acid served as the matrix.

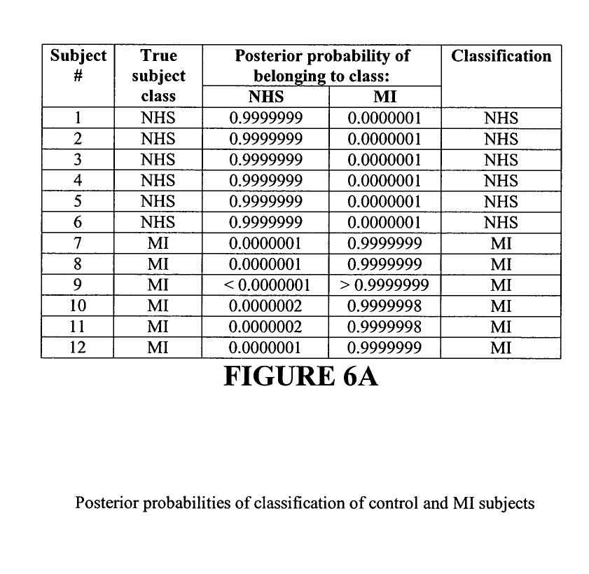

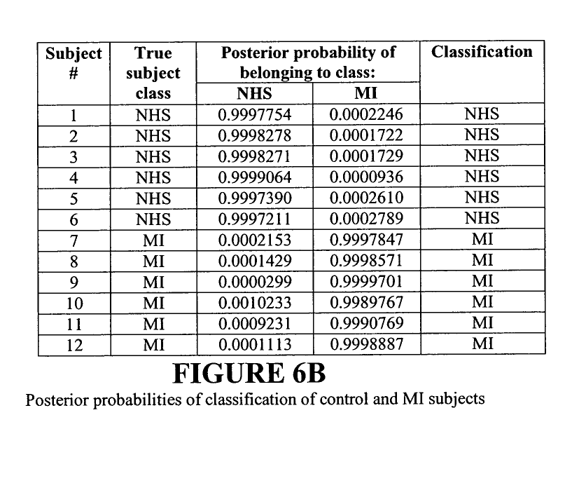

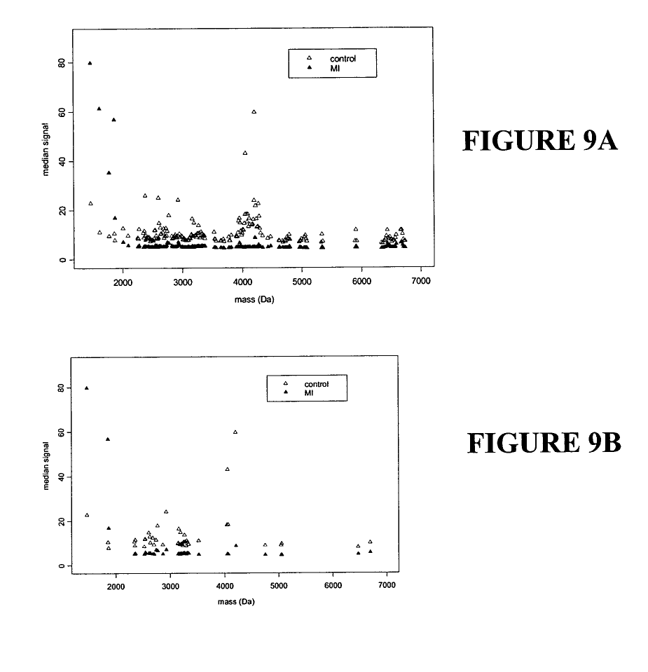

A combination of the fundamental principals in decision tree analysis (Qu et al. Clinical Chemistry 48(10):1835-1843 2002) and multivariate analysis (Fung et al. Biotechniques Supplement 34-38, 40-41 2002; Li et al. Clinical Chemistry 48(4):1296-1304 2002) was used to analyze the spectra presented in FIG. 8. When a significance level of a=0.001 was utilized, a total of 45 windows were identified within which the difference in mean signal strength between control sera samples and MI sera samples was deemed statistically significant. FIG. 9A displays the median signal strengths with respect to both control samples and MI samples for each of these windows. The distribution of peptides between normal and MI serum that differ in median intensity by an average of two fold. The data comprising the spectra shown in FIG. 8 plus additional spectra were collected in numerical form with signal strength corresponding with mass. FIG. 9A shows median signal strengths corresponding with mass levels showing significant differences between controls and MI subjects at a significance level of a=0.001. The results of the discriminant analysis are displayed in the tables of FIGS. 6A-B; all twelve subjects were correctly classified with a minimum posterior probability of 99.9999%. When a significance level of a=0.005 was utilized, 200 windows were identified within which the difference in mean signal strength between control sera samples and MI sera samples was deemed significant. FIG. 6b shows median signal strengths corresponding with mass levels showing significant differences between controls and MI subjects at a significance level of a=0.005. By applying the discriminant analysis on the data within these windows, all twelve subjects were correctly classified with a minimum posterior probability of 99.89%. It is interesting to note that by employing a higher significance level, the dimensionality of the retained data set increased four-fold, with no concomitant increase in the predictive power of the resulting discriminant analysis. The statistical techniques employed here appear to provide a powerful methodology with respect to the correct classification of MI and control subjects.

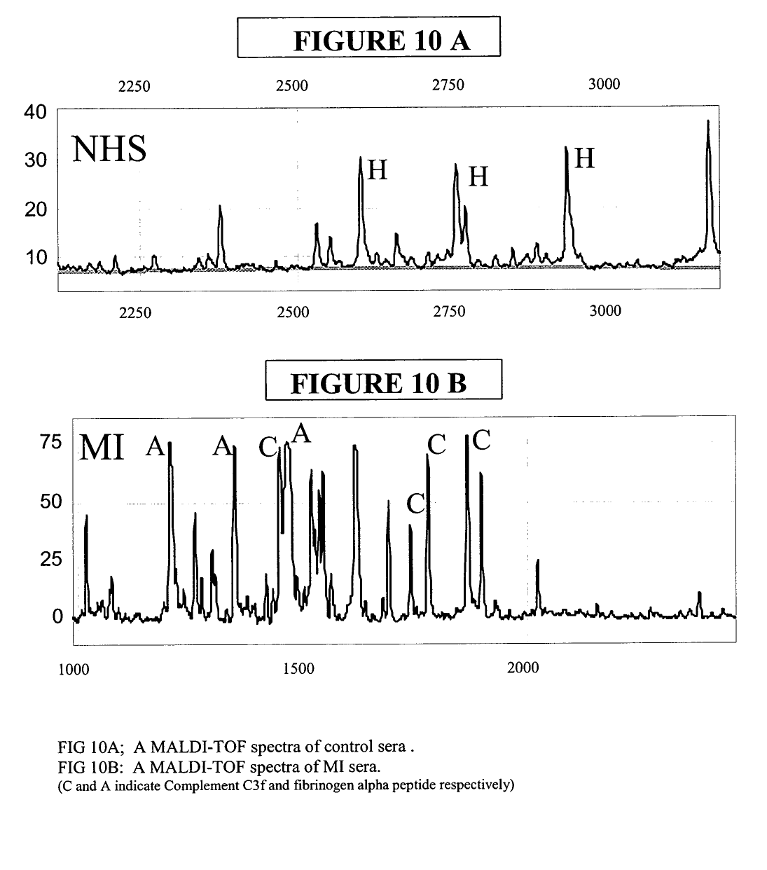

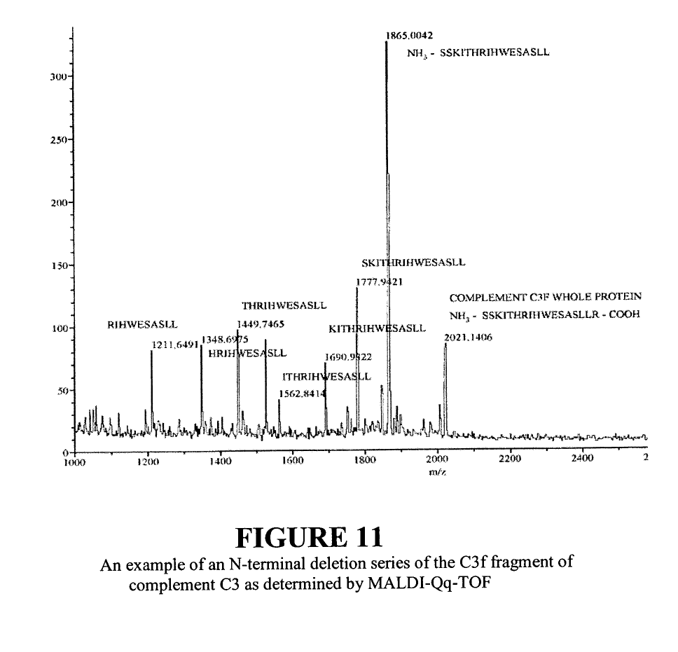

The distinctive peaks in NHS and MI sample were sequenced using MALDI-Qq-TOF (Loboda et al. Rapid Commun Mass Spectrometry 14(12):1047-1057 2000; Shevchenko et al. Analytical Chemistry 72(9):2132-2141 2000). It was found that the peaks observed in NHS and MI samples were fragments of common sera proteins. Peptides from the common sera proteins human sera albumin were observed in the control(reference) spectra (FIG. 10A). FIG. 10A shows the identity of peptides in the control and MI sera as determined by a MALDI-Qq-TOF using an interface to a Micro Mass mass spectrometer related to MALDI TOF spectra obtained with the Ciphergen Biosystems PBSII. FIG. 10A shows MALDI-TOF spectra of control sera showing the identity of the peptides. The H indicates the peptide is derived from Human Serum Albumin. The family of peaks observed in MI samples were comprised by the C3f fragment of complement C3b alpha chain and C3f fragment progressively missing amino acids from the N terminus and some fragments of alpha fibrinogen. FIG. 10B shows the MALDI-TOF spectra of MI sera showing the identity of the peptides. The letters C and A indicate that the peptide is derived from Complement C3f and fibrinogen alpha peptide respectively. In fact we even observed and MS/MS analysed each member of a near perfect ladder of C3f in the sera of a heart attack patient by MALDI-Qq-TOF. FIG. 11 shows an example of an N-terminal deletion series of the C3f fragment of complement C3 as determined by MALDI-Qq-TOF. Note that the parent C3f fragment is produced by a tryptic like cleavage on the N-terminal side of arginine but occasionally with cleavage on the C-terminal side of arginine. The C3f fragment shows the progressive loss of amino acids from the N-terminal end. The MS/MS analysis of the peptides showed the full C3f fragment SKITHRIHWESASLLR (SEQ ID NO:10) and the loss of residues from the N-terminus resulting in a ladder of fragments. The smallest fragment observed was RIHWESASLL (SEQ ID NO:2). MS/MS fragment patterns were obtained for each member of the family of peaks observed by MALDI-MS and searched these against the NCBI and Swiss Pro data bases to confirm their identify as sub-fragments of C3f. However, it is also noted that in this case of a progressive loss of single amino acids from the N termini some sequence can be called directly from the MS spectra. Thus it was found that the C3f fragment of complement C3 was released, presumably by the action of a serine centered endoproteinase (Volanakis Current Topics in Microbiology and Immunology 153:1-21 1990; Fishelson Molecular Immunology 28(4-5):545-552 1991). However, it was also observed that the released C3f fragment was apparently degraded from the N-terminus by the action of an exopeptidase similar to the aminopeptidases previously described in human sera (Sanderink et al. Journal of Clinical Chemistry and Clinical Biochemistry 26(12):795-807 1988).

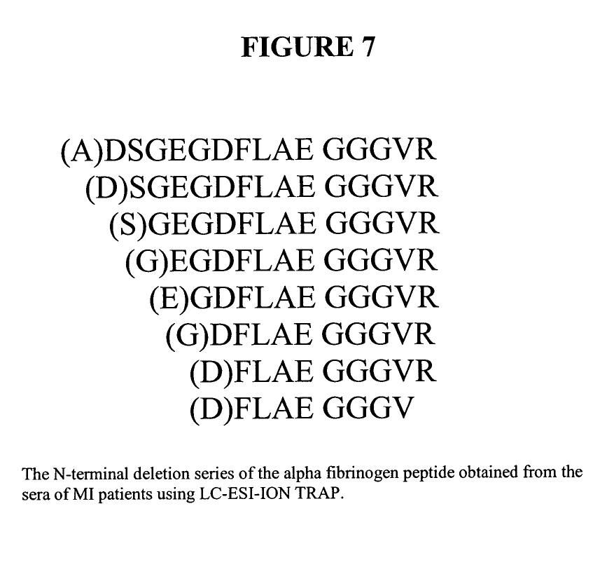

While most of the interest in sample profiling to date has been in the use of MALDI-TOF (Oleschuk et al. Biomaterials 21(16):1701-1710 2000) it is noted that other mass spectral devices might be used to generate data that could be used for comparison. On comparison of MI verses normal sera samples on LC-ION TRAP using a pattern matching algorithm; the presence of peptides of alpha fibrinogen and C3 was also observed in the sera MI patients. For example, it was found that the presence of the compliment C3f fragments and N-terminally truncated C3f in the MI sample [SSKITHRIHWESASLL (SEQ ID NO:8), THRIHWESALL (SEQ ID NO:11), and IHWESLL (SEQ ID NO:12)] but we also found other fragments of complement C3 (T)MSILDISMMTGFAPDTDDLK (SEQ ID NO:13) and (S)HVSELLML (SEQ ID NO:14] in MI but not in the normal sera sample. In addition, a near perfect ladder of N-terminal deletions peptides of alpha fibrinogen was observed (FIG. 7). Thus the LC-ESI-ION TRAP analysis showed the presence of a ladder of fibrinogen peptides that, similar to C3f, had apparently also been degraded by a N-terminal exopeptidase, i.e. aminopeptidase (Sanderink et al. Journal of Clinical Chemistry and Clinical Biochemistry 26(12):795-807 1988). Hence agreement was observed on the presence and identity of C3f and fibrinogen alpha peptide in the serum of MI patients with both MALDI and ESI based spectrometry: Both methods agreed that C3 peptides and alpha fibrinogen were found predominantly in the sera of MI patients but not controls (references) and both methods agreed on the presence of N-terminally deleted peptide fragments (Griffin et al. Analytical Chemistry 73(5):978-986 2001).

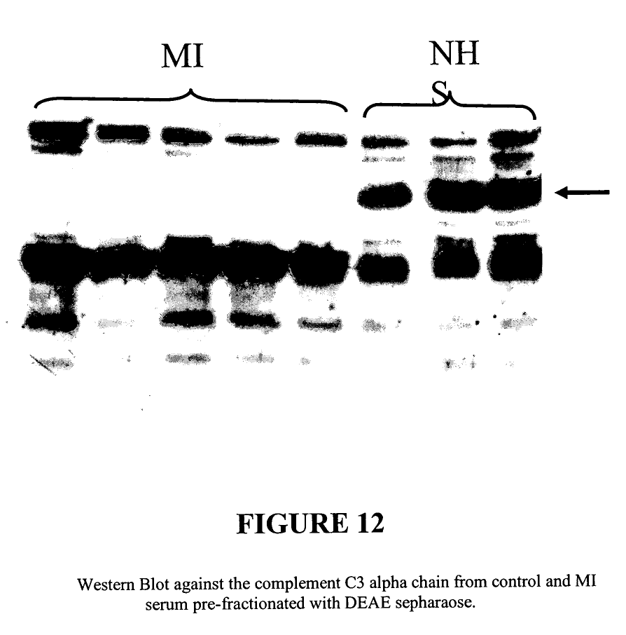

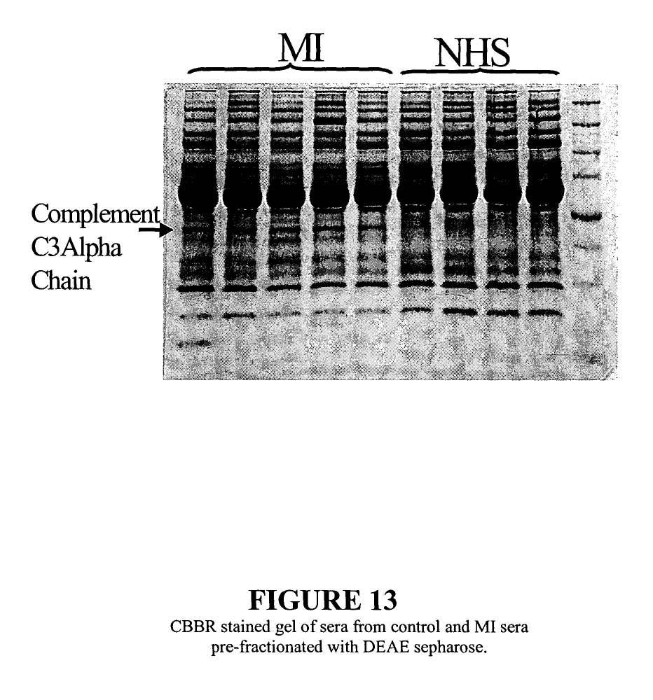

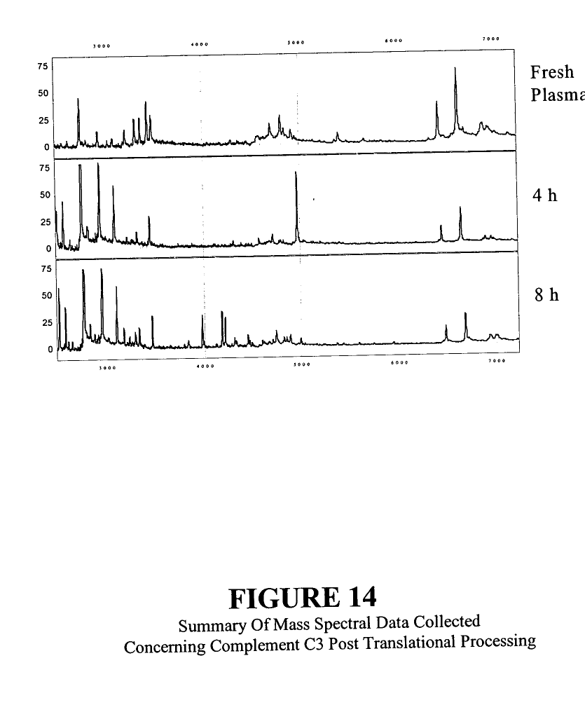

Since C3f is derived from the proteolytic degradation of the complement C3 alpha chain, the generation of the C3f fragment in MI samples indicates that the proteolytic procesesing of C3b alpha had occurred. To confirm this prediction a western blot analysis was performed with a mono-clonal antibody against the C3b alpha chain. It was determined empirically that DEAE pre-fractionation of sera yielded the clearest picture of C3 alpha chain proteins when gels were visualized by western staining. It was confirmed that alternative proteolytic processing of C3b occurred in MI patients using a Western blot against the DEAE fraction with an anti C3b antibody. In normal human sera it was found that the complement C3 chain was typically fragmented into three main polypeptides with relative molecular masses of approximately 125, 70 and 40 kD. FIG. 12 shows a western blot against the complement C3 alpha chain from control and MI serum pre-fractionated with DEAE sepharaose. Five typical MI and 3 typical control of six serum samples are shown. The precursor complement C3 molecule, the full length alpha chain (control) and a processed form the C3 alpha chain (predominate in MI) are apparent. The arrow shows the location of the 70 kD processed form in control samples in FIG. 12. In contrast, in MI sera the polypeptide with mass of 70 kD was essentially missing while the protein product with a mass of about 40 kD was more intense. FIG. 13 shows the presence of a fragment of C3 alpha displaying a relative molecular mass of about 40 kD as detected by SDS-PAGE. FIG. 13 shows a CBBR stained gel of sera from control and MI sera pre-fractionated with DEAE sepharose. The sera were resolved by SDS-PAGE prior to staining with CBBR 250. The location of the band sequenced by MALDI-Qq-TOF and LC-ESI-ION TRAP is shown by an arrow. The CBBR stained gel reveals an apparently greater amount of the 40 kD band. It is noted that CBBR staining is more quantitative the ECL western blots. The identity of this band was confirmed as a fragment of C3 alpha by MS/MS fragmentation by both MALDI-Qq-TOF and LC-ESI-ION TRAP. The result of the peptide coverage obtained with respect to the complement C3b sequence by both LC-ESI-ION TRAP and MALDI-Qq-TOF are presented in FIG. 14. FIG. 14 shows a summary of mass spectral data collected concerning complement C3 post translational processing. The complement C3f fragment (aa1303 to aa1320) was detected by both MALDI-Qq-TOF and LC-ESI ION TRAP. The portion of the C3 alpha chain C-terminal to the C3f fragment was resolved by SDS-PAGE and five peptides were identitified by MALDI-Qq-TOF and ESI ION TRAP. The peptide coverage of this C3 alpha fragment is intense on the C terminal side of the C3f fragment site but no sequences were detected on the N-terminal side of the C3f fragment. Moreover, the mass of the protein product is also consistent with the C-terminus of C3 that would be released after proteolytic cleavage and release of C3f. Hence it was found that this band is comprised of the bulk of C3 alpha from the C3f site to the C-terminus of the molecule.

As noted above, upon MS/MS analysis of the main polypeptide peaks of less than 3000 D in normal human sera with a MALDI-Qq-TOF it was also found that the main peptides were proteolytic fragments of commonly abundant sera proteins such as human serum albumin and others. Since the distinctive pattern in MI patients was derived from complement and fibrinogen fragments and those of the control were from other abundant proteins such as serum albumin, the hypothesis was examined that the astonishing utility of the disease specific SELDI profiles resulted from the differential post-translational modification of common blood proteins that uniquely reflect each physiological state. Furthermore, the instant inventors attempted to reveal the mode by which these diagnostic patterns were formed.

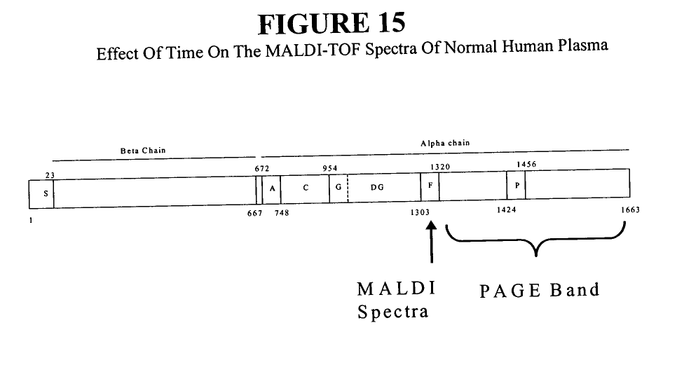

One of the initial questions posed was to determine whether these fragmentation patterns exist in vivo or if they are generated ex vivo. Since sera by its nature is an ex vivo artifact produced by the activation of the proteolytic coagulation cascade, for the initial experiments freshly drawn plasma or plasma left on the bench for various lengths of time was used. It was observed that absolutely fresh plasma does not show the characteristic family of peptides that were found in plasma after sitting at room temperature for 4h (FIG. 15). FIG. 15 shows the effect of time on the MALDI-TOF spectra of normal human plasma. Venous blood was collected into citrated tubes and centrifuged at 14,000 g for 30 seconds before the plasma was collected and immediately (Time 0), or left on the bench at 25 degrees celcius for the time indicated prior to, before dilution 10 fold in 0.1% TFA in water followed batch collection of the peptides by reversed phase C18 chromatography. The sample was analyzed at a laser intensity of 210 and a sensitivity of 7 on a Ciphergen Biosystems PBSII. The result of one experiment representative of three is shown. The pattern of fragments in plasma changes within as little as 2 h at room temperature with some of the larger polypeptides apparently lost from the spectra and some lower mass peptides apparently increasing in intensity. The altered peptide pattern once formed changes slowly overt time and is still recognizable after 72. The pattern also changed with storage at 4 degrees celcius, albiet more slowly and up to 3 freeze/thaw cycles did not have a marked effect on the pattern. Thus it was observed that the patterns observed in plasma apparently were generated ex vivo and generally stable for an extended period of time once formed. These changes in the plasma profile soon after removal of the blood from the body indicate that some process occurs in the blood ex vivo that influences the peptides spectra. The most obvious possibility to pursue was the role of proteases.

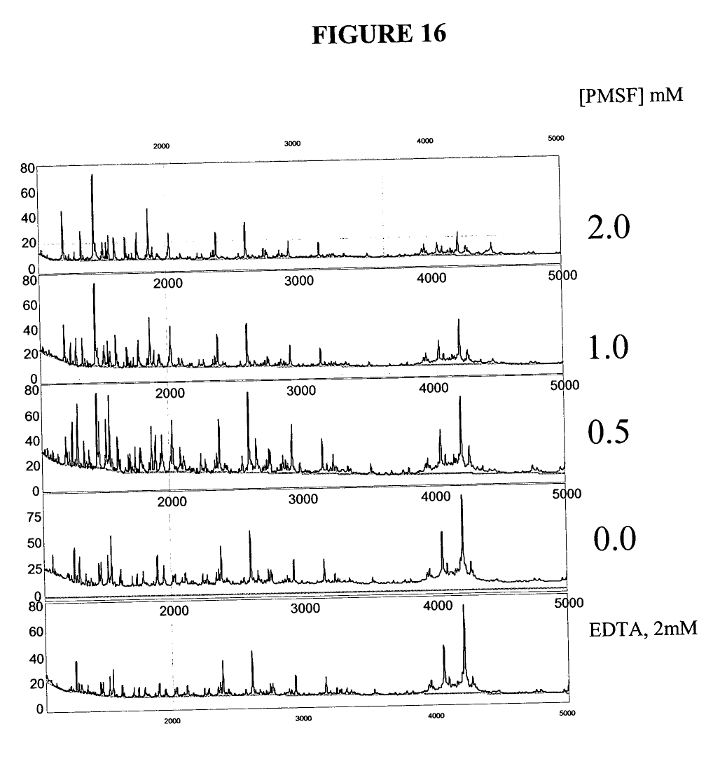

In order to establish a role for proteases in the formation of the diagnostic peptide pattern in sera that has already undergone the activation of the proteolytic coagulation cascade, the broad spectrum serine-centered protease inhibitor PMSF and the iron sulphur protease inhibitor EDTA were employed. It was observed that incubation of sera samples with EDTA had no effect on the distribution of peptides in the mass spectra by MALDI-TOF (FIG. 16). FIG. 16 shows the effect of the iron-sulphur protease inhibitor EDTA and the serine-centered protease inhibitor PMSF on the MALDI-TOF spectra of normal human sera as detected by MALDI-TOF. Crystals of PMSF or the sodium salt of EDTA were added directly to 1 ml of sera and incubated for 4 h before before sampling. At the time of sampling a 25 micro litre aliquot of sera was diluted ten fold in 0.1% TFA before collection of peptides by batch C18 reversed phase chromatorgraphy and spotting on gold MALDI-TOF targets. The sample was analysed at a laser intensity of 190 and an amplifier sensitivity of 7 on a Cyphergen Biosystems PBSII. The result of one experiment representative of three is shown. The arrows show the location of newly appearing bands in PMSF-treated sera. However, a dose-dependant effect of PMSF on the pattern of polypeptides in the MALDI-TOF spectrum was observed. Concentrations of PMSF in the micromolar range has little effect on the peptide pattern but concentrations of 1 mM PMSF or greater produced a dramatic reduction in signal strength of higher mass polypeptides and an increase in the apparent complexity of lower mass peptides. Thus, the peptide distribution across the sera mass spectrum could be perturbed by the inhibition of serine centered proteases. Hence it was found that the perturbation of the peptide profile by PMSF indicates that the profile results in part from the action of endopeptidases.

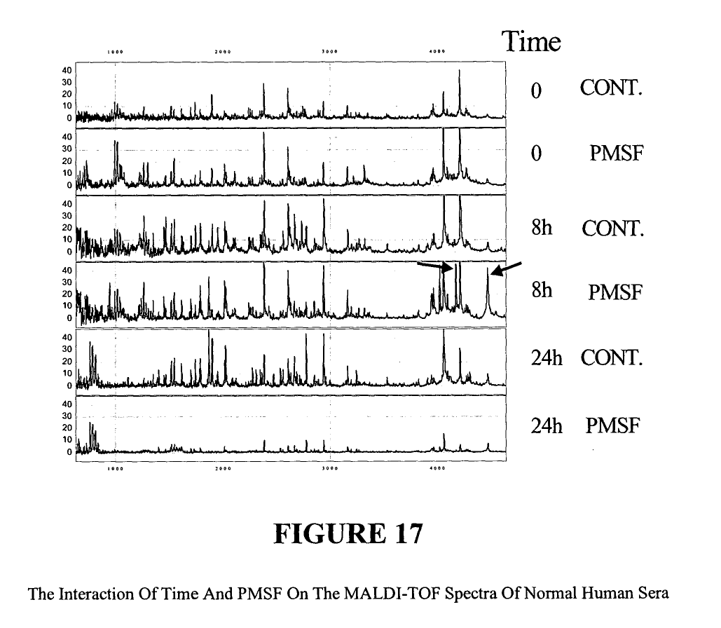

The effect of time on the peptide profile and the interaction of time and serine proteinase inhibition was also examined. It was found that the profile of sera changed with time on incubation at room temperature (FIG. 17). FIG. 17 shows the interaction of time and PMSF on the MALDI-TOF spectra of Normal human sera. One ml aliquots of human sera were thawed and instaneously treated with PMSF or nothing. Twenty five microlitre aliquots were then immediately (Time O), or left on the bench at 25 degrees celcius for the time indicated before being, diluted ten fold in 0.1% TFA and the peptides collected by batch reversed phase chromatography, eluted onto gold MALDI targets matrixed with CHCA and analyzed at a laser energy 210 and a sensitivity of 7 on a Ciphergen Biosystems PBSII. The result of one experiment representative of three is shown. After about 8 h of incubation significant alterations in the peptide profile were observed in sera. These differences became even more pronounced after 24 h of incubation at room temperature. These results indicated that sera was unstable and that the increasing complex profile is a direct reflection of the degradation with time and presumably proteolytic activity. This observation was reinforced by the interaction of time with serine proteinase inhibtor treatment. No difference was observed in normal sera immediately after the addition of the PMSF. However, within as little as 2 h, and typically between 4 and 8h of incubation, PMSF showed distinct effects on the pattern of peptide mass distribution resulting in the appearance of some higher molecular mass peptides. A strong interaction between time and PMSF treatment was observed within 24 hours when the failure of PMSF treated sera to generate peptide fragments via endopeptidase activity apparently resulted in a generalized loss of peptides from the MALDI-TOF spectrum. In untreated sera about 72 h of degradation was required before the sera sample was run down and no longer produced peptides in the low molecular mass range.

The requirement for time to reveal the effects of PMSF indicates that the change in the spectra is not a direct result of the presence of PMSF but was the result of the inhibitors' influence on an enzymatic activity during the course of incubation. In addition to the control of adding PMSF to sera immediate before processing, acidified sera was incubated with PMSF for several hours or added PMSF directly to boiled sera before incubation to ensure that the loss of peptides from the mass spectra was not due to any inhibitory effect of PMSF on the MALDI ionization process. Moreover, it was confirmed that the general loss of low mass peptides in PMSF treated samples was by rp-LC-ION TRAP. Thus it was observed that the effect of PMSF in reducing peptide complexity and signal strength did not result from a contaminating effect of PMSF on the MALDI process but seemed to result from the inhibition of endo-proteolytic cleavage by serine proteases. Hence the instant inventors found the low molecular mass polypeptide patterns in human sera to be dependant on the activity of proteases.

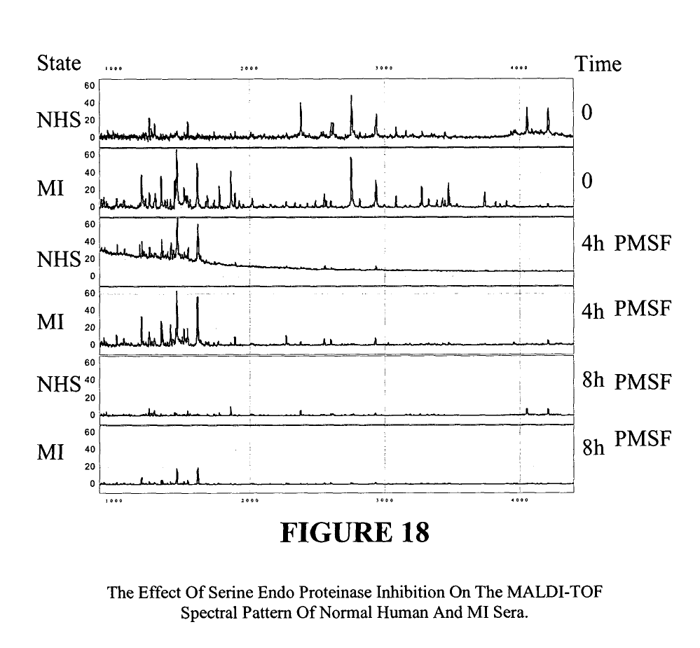

The distinctive patterns of peptides on both normal human sera and MI sera were found to be sensitive to PMSF over time. Before the addition of PMSF the normal human sera and MI sera showed different distributions of polypeptides (FIG. 18). FIG. 18 shows the effect of serine endo proteinase inhibition on the MALDI-TOF spectral pattern of normal human and MI sera. Sera was sampled immediately or left on the bench after treatment with PMSF for the time indicated before it was diluted in acid, the peptides collected by batch reversed phase chromatography, eluted onto gold MALDI targets, matrixed with CHCA and analysed at a laser intensity of 210 and a sensitivity of 7 with a Ciphergen Biosystems PBSII. The result of one experiment representative of three is shown.

However, upon addition of the serine-centered endo-peptidases inhibitor PMSF the profiles appeared remarkably similar with several hours: Hence it was found that the activity of serine centered protease was responsible for the different peptide profiles between normal and MI sera. With extended incubation time, adding PMSF to normal human sera resulted in a loss of most low molecular weight peptides from the MALDI-TOF spectra (FIG. 18). Similarly, the addition of PMSF destroyed the pattern of the peptides found in both NHS and MI by about 8h the samples were virtually devoid of low molecular weight peptides as assayed by MADLI-TOF. Thus the addition of PMSF ablated the differences in spectra between the control and disease state leaving only common elements within a few hours and further incubation essentially erased most of the spectral elements. From these data it was found that serine centered endoproteinases were responsible for the distinctive MALDI-TOF spectra of normal and MI sera.

In particular, it was found that preparing sera by rapid pre-separation over C18 reversed phase resin prior to MALDI-TOF resulted in strong signals, excellent signal to noise ratio, reproducible spectra and sharp statistical resolving power. These results indicate that many diseases and specific variants may well be distinguishable by rapidly performed mass spectral assays that do not require disease-specific-reagents.

The observation that the patterns in sera or plasma depend on how the sample has been collected and stored leads to a practical consideration in the coming world-wide effort to characterize the proteome of human blood (Hanash et al. Molecular Cell Proteomicsl(6):413-4414 2002). One of the most important standards set in the Human Proteome Organization (HUPO) congresses scheduled for this year should be the standardization of sample collection procedures. In terms of sera profiling for disease, it is recommended that standards should be set for time and temperature at which blood is clotted, the conditions of centrifugation, the time sera remains unfrozen and how it is aliquoted, frozen thawed and used. Without such standards it is apparent that it will be impossible to meaningfully compare the results obtained in one laboratory with those of another. It is recommended that sera be coagulated at room temperature for 2h before clinical centrifugation for 20 minutes and the sera frozen at the clinical site. After shipping on dry ice the sample may be thawed once for aliquoting at the laboratory site, re-frozen and the aliquots subsequently used once and discarded. Of course it matters little which reasonable standard is adopted as long as one reasonable standard is adopted.

It was found that low molecular weight families of polypeptides can be used to distinguish between control and MI patients. If it is envisaged that each disease is associated with damage or death of specific cells or organs then it is not unreasonable to assume that the disease process will result in the differential release, secretion or activation of different enzymes from the affected cells or in response to the damage cells. If different post-translational modification activities or specificities are manifest with each disease and have different affinities for the major proteins in the blood then it is not difficult to image that that each different disease might be associated with different post translational modifications of major blood proteins. The concentration of proteins released into the blood directly from the damage cells, or the changes in potent regulatory factors associated disease, are likely to be far too small to be directly detected by MALDI-TOF. Therefore, it is not reasonable to assume that the altered peptide spectra are a direct measure of disease proteins. Rather, the changes in the spectra seem to reflect the action of disease-associated enzymatic activity or specificity on major blood proteins. Hence, the data here indicates that differential reactions of major blood proteins with disease-associated enzyme activities is the most tenable explanation for the phenomina of disease specific MALDI-TOF spectra at least in the case of normal sera verses MI sera. Consistent with this concept, the success of the IMAC chip for profiling cancer patients (Paweletz et al. Disease Markers 17 (4):301-307 2001; Ardekani et al. Expert Rev Mol Diagnostics 2(4):312-320 2002; Li et al. Clinical Chemistry 48(8):1296-1304 2002) may reflect the the capacity of IMAC chromatography to bind proteins that have been post-translationally modified with phosphate groups (Andersson et al. Analytical Biochemistry 154(1):250-254 1986; Muszynska et al. Journal of Chromatography 604(1):19-28 1992).

In this regard, the low molecular mass polypeptides found in blood of MI sera seem to result from proteolyic cleavage of C3, a major protein component of blood. The appearance of these patterns in the sera indicate the presence of functional endo peptidases in blood that generate peptide fragments of from major serum or plasma proteins. The loss of these peptide fragment patterns ex vivo with the addition of PMSF confirms the presence of serine centered endo-peptidases but also indicates that endo peptidases are constantly generating peptide fragments from major proteins in sera and plasma samples over time. However, the loss of peptide spectra with time after the addition of PMSF also indicates that N-terminal exopeptidases are constiutively cleaning up the products of serine proteinase in blood. Most importantly, these two processes seem to be in balance with one another to such a degree that a brief pertubation with PMSF results in the overall loss of detectable peptides. In the case of normal human sera and MI sera the peptides that discriminate between these two physiological states were apparently generated ex vivo by the action of endo and exo proteinases. However it is emphasized that the generation of the peptides ex vivo in both normal serum and MI sera in no way diminished their utility as diagnostics.

The conclusion that both endo and exoproteolytic activities form the diagnostic pattern was examined in more detail using time course and inhibitor studies. Since the distinctive pattern of normal plasma was not present in plasma immediately after collection but was generated with time it can be infered that the activity of endoproteinases generated the fragment patterns in sera in vitro after collection of the blood. Experiments in sera demonstrating that the effect of PMSF was dependant on both dose and time support the view that serine-centered endoproteinases are responsible for the diagnostic peptides patterns. Moreover, since the pattern once established, is relatively stable for some time it may indicate that peptides were being constitutively generated in sera samples after collection. The dose required to prevent the pattern, =2 mM, closely matches the dose of PMSF commonly used to prevent proteolysis. If the effect of PMSF was derived from some chemical interference with the MALDI process then it might be expected that it should show an inhibitory effect in the sub-millimolar range and show its effect immediately upon addition and not require several hours to ablate the profile. Hence, from the appearance of peptides after collection of the plasma and effect of PMSF on sera with time it is concluded that the distinctive patterns generated in normal human sera and MI sera result from the activity of serine centered proteinases ex vivo.

Since adding PMSF, which initially inhibited the formation of the high mass peptides coupled to the accumulation of low mass peptides, eventually resulted in the erasure of distinctive pattern of peptides it can be concluded that this is evidence that exopeptidases are constantly degrading peptides in the blood. The eventual loss of the distinctive family of peptides upon addition of PMSF indicates that exo-peptidases remain functional in sera and plasma and are constantly degrading the peptides that form the diagnostic pattern. Thus the distinctive patterns of <10 kD peptides observed in normal and MI sera samples represent the balance between the effect of endo and exo-proteases. Similarly, the observation of the degradation of C3f or the alpha fibrinogen peptide showing the progressive loss of amino acids from the N terminus thus producing a family of fragments was also consistent with the activity exo-peptidases. The ladder of peptides showing the progressive loss of N-terminal amino acids alone is sufficient to conclude that the patterns in MI must result from the balance of and endoproteotyltic activity generating the parent fragment and exoproteolutic activities degrading the full length peptide. Hence the success of diagnosis by MALDI patterns may extend to those diseases that cause a significant change in balance of concentration or activity proteases in the serum. Hence it appears that at least in the case of normal and MI sera MALDI-TOF peptide pattern diagnosis works by comparing the mass distribution of peptides and their associated N-terminal exoproteolytic products. The measurement of these forms the basis of the rapid and unambiguous mass spectral diagnosis for MI that requires no specialized reagents.

The differential processing of complement C3 alpha chain in normal human sera verses MI sera may yield important clues for the therapeutic prevention of damage to the heart in response to MI. Animal models of myocardial infarction activated the complement system in rats and in human MI patients, the components of the classical complement cascade including C3 were associated with the membranes of necrotic tissues (Frangogiannis et al. Cardiovascular Research 53(1):31-47 2002). The mRNA and proteins of all the components in the classical pathway have been shown to be up-regulated in myocardial infarcts (Vakeva et al. Circulation 97(22):2259-2267 1998; Yasojima, Schwab et al. Circulation Research 83(8):860-869 1998). The chemotactic activity of post ischemic cardiac lymph that may recruit monocytes and neutrophils was inhibited by neutralizing antibodies to the complement activating factor C5a (Dreyer et al. Circulation Research 71(6):1518-1524 1992; Birdsall et al. Circulation 95(3):684-992 1997). While there is evidence that post ischemic damage to the heart is mediated by the complement system, the blanket inhibition of the complement cascade is not likely to benefit heart attacks patients in the long term. Systemic inhibition of the inflammatory pathways using corticosteroids actually increased the damage done by myocardial infarction (Frangogiannis et al. Cardiovascular Research 53(1):31-47 2002). However, more targeted depletion of complement reduced the size of myocardial infarcts (Maroko et al. Journal of Clinical Investigation 61(3):661-670 1978). Similarly, infusion of soluble human complement receptor type 1 (sCR1) decreased the size of infarct in a rat model (Weisman et al. Science 249(4965):146-151 1990; Weisman et al. Trans Assoc Am Physicians 103:64-72 1990). Hence detailed knowledge of the mechanisms whereby the proteolytic machinery is activated and functions in myocardial infarction will be required to design practical therapies that precisely target the products of the of the complement system that cause injury without preventing the role of complement in tissue healing (Frangogiannis et al. Cardiovascular Research 53 (1):31-47 2002).

Here it is shown that enzymes capable of releasing the C3f fragment of complement C3 alpha and the alpha peptide from alpha fibrinogen are active in the blood of MI patients ex vivo. Moreover, the complement processing activities in the MI sera are capable of cleaving the C3 alpha chain to produce the C3f fragment and thus also releasing the C terminal portion of the protein. The presence of the C3f fragment, which has not been previously associated with MI is of course a natural corollary of complement activation. Similarly, the activation of fibrinogen fragmentation has also been previously connected to MI (Kaplan et al. Heart Disease 3 (5):326-332 2001; Gil et al. International Journal of Cardiology 83 (1):43-46 2002). There is considerable evidence to link the activation of the complement system with MI and this is consistent with the consequent generation of C3f. Hence the spectral pattern of the heart attack reflects the peptide generated by these two well established proteolytic pathways associated with MI and illuminates some of the major mechanisms associated with disease. From this it is hopeful that the proteomic characterization of proteins and peptides released into the blood by other less characterized diseases will likewise reflect the molecular mechanisms associated with disease and may well yield important clues for possible treatments.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for diagnosing and distinguishing a myocardial infarction in a particular human subject by comparing a serum protein profile of said particular human subject to the reference serum protein profiles of at least two or more defined subsets of human subjects wherein said serum protein profiles are generated by mass spectrometry comprising the steps of;
(a) identifying areas of said serum protein profiles that are different in signal intensity between the particular human subject and the defined subsets of human subjects wherein the difference in signal intensity represents a difference in protein mass;
(b) reducing the dimensionality of the areas identified in step(a) in order that the signal intensities associated with protein masses identified in step (a) are retained for each particular subject;
(c) elucidating a metric in order to identify a particular human subject; and
(d) analyzing said metric elucidated in step (c) in order to identify a particular human subject based upon statistical comparison of characteristics of the reference serum protein profiles of human subjects belonging to the said two or more defined subsets of human subjects, whereby a myocardial infarction is diagnosed and distinguished in said particular human subject.
